(19) United States
(12) Patent Application Publication
(10) Pub. No.: US 2010/0145258 A1
(43) Pub. Date: Jun. 10, 2010

(54) SYSTEMS AND METHODS FOR ELIMINATING POST-EXCITATION VIBRATION IN OPHTHALMIC SURGICAL HANDPIECES

(76) Inventor: David Hertweck, Valley Park, MO (US)

Correspondence Address: Bausch & Lomb Incorporated, One Bausch & Lomb Place, Rochester, NY, 14604-2701, US

(21) Appl. No.: 12/329,884

(22) Filed: Dec. 8, 2008

(51) Int. Cl. A61F 9/007

(52) U.S. Cl. 604/22; 606/107

(57) ABSTRACT

A method of eliminating post-excitation vibration provided from a surgical handpiece to a patient's eye during ophthalmic surgery. The surgical handpiece includes an ultrasonic vibration structure for generating vibration and a needle for transmitting the vibration into the patients eye. The method includes exciting the ultrasonic vibration structure and thereby causing the needle to vibrate for emulsifying tissue in the patient's eye, ceasing the excitation of the crystal structure, and generating a brake signal to restrain the vibration of the needle, upon ceasing the excitation of the crystal structure, thereby reducing the vibration provided to the patient's eye after excitation of the crystal structure has ceased.

### BACKGROUND

1. Field

The present disclosure is directed to systems and methods for eliminating post-excitation vibration in ophthalmic surgical handpieces.

2. Description of the Related Art

The statements in this section merely provide background information related to the present disclosure and may not constitute prior art.

In eye surgery, particularly phacoemulsification (phaco) surgery, ultrasonic vibration is commonly used to emulsify tissue within an eye of a patient. Ultrasonic vibration is often provided by a stack of crystals included in a surgical handpiece having a needle connected thereto. Initially, an operator inserts the needle into patient's eye. By applying a voltage to the crystals, in response to an operator request, ultrasonic vibration is produced. The ultrasonic vibration is delivered to an eye through the needle. When an operator no longer requests ultrasonic vibration, the voltage is removed from the surgical handpiece, and specifically, the crystals. When the voltage is removed, the crystals continue to vibrate for an interval, commonly understood as a “ring-down” interval of the crystals. During the “ring-down” interval, the needle, which remains inserted in the eye, continues to deliver ultrasonic vibration to the patient's eye. In some instances, a period between multiple applications of voltage to the crystals may be less than the “ring-down” interval, such that the crystal constantly vibrates the needle irrespective of request from the operator.

In order to minimize an amount of ultrasonic vibration delivered to an eye during ophthalmic surgery, it is desirable to provide vibration to the patient's eye only when requested by an operator of a surgical handpiece.

### BRIEF DESCRIPTION OF THE DRAWINGS

The drawings described herein are for illustration purposes only and are not intended to limit the scope of the present disclosure in any way.

FIG. 1 is a block diagram of a method of eliminating post-excitation vibration provided from a surgical handpiece according to the present disclosure;

FIG. 2 is a block diagram of an ophthalmic surgery system including a surgical handpiece according to the present disclosure;

FIG. 3 is a waveform diagram illustrating an excitation signal and a brake signal; and

FIG. 4 is a block diagram of an ophthalmic surgery system including a surgical handpiece having a brake mechanism.

### DETAILED DESCRIPTION OF THE PREFERRED EMBODIMENT

The following description is merely exemplary in nature and is not intended to limit the present disclosure, application, or uses.

According to one exemplary embodiment of the present disclosure, a method 100 of eliminating post-excitation vibration provided from a surgical handpiece to a patient's eye during ophthalmic surgery is illustrated in FIG. 1. The surgical handpiece typically includes a crystal stack structure for generating vibration and a needle for transmitting the vibration into the patient's eye. It is to be understood that the present example is being shown using a piezo-resistive handpiece, but may equally apply to magneto-resistive handpieces as well. Method 100 includes step 102 for exciting the crystal structure and causing the needle to vibrate for emulsifying tissue in the patient's eye. For phacoemulsification surgery, the needle is vibrated at ultrasonic frequencies to effectively emulsify a cataract. The method 100 also includes step 104 for ceasing the excitation of the crystal structure and step 106 for generating a brake signal to restrain the vibration of the needle, upon ceasing the excitation of the crystal structure, thereby reducing or eliminating the vibration provided to the patient's eye after excitation of the crystal structure has ceased.

By utilizing a brake signal to eliminate vibration of the needle after ceasing excitation, method 100 allows the operator to minimize the amount of vibration transmitted to the patient's eye over an entire ophthalmic surgery and to apply ultrasonic energy to the eye only when desired. In one example, during a phacoemulsification procedure, an operator may excite a crystal structure to cause ultrasonic vibration multiples times during an ophthalmic surgery, and therefore, also cease excitation of the crystal structure multiples times in the ophthalmic surgery. Over the ophthalmic surgery, the amount of ultrasonic vibration transmitted to the patient's eye during “ring-down” may be eliminated by generating a brake signal according to the present disclosure for each of the multiple cessations of excitation of the crystal structure.

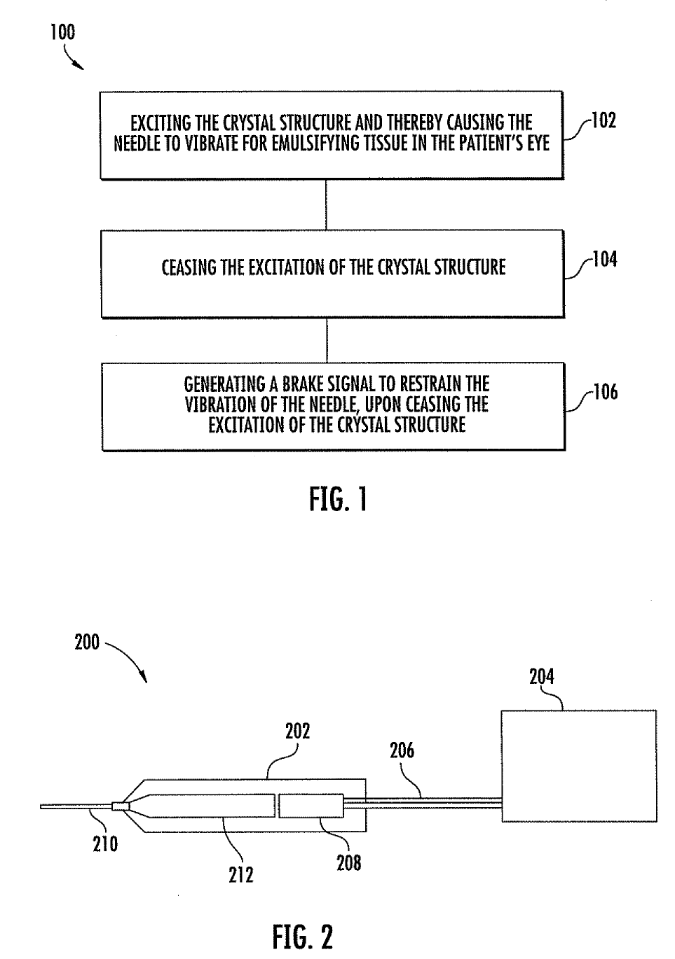

The method illustrated in FIG. 1, and described in the present disclosure, may be implemented in a number of different surgical handpieces and/or ophthalmic surgery systems for eliminating post-excitation vibration provided from a surgical handpiece to a patient's eye during ophthalmic surgery. One exemplary embodiment of an ophthalmic surgery system for implementing the methods described herein is illustrated in FIG. 2 and generally referenced 200.

The ophthalmic surgery system 200 includes a surgical handpiece 202 and a control circuit 204, which is electrically coupled to the surgical handpiece 202 via cable 206 The surgical handpiece 202 includes an ultrasonic vibration structure 208, a needle 210, and a horn 212. The ultrasonic vibration structure 208 will be illustrated in this example as a crystal structure 208, but other structures may be used The crystal structure 208 is in mechanical communication with the needle 210 through the horn 212. In this particular embodiment, the crystal structure includes two piezoelectric crystals. It should be appreciated that a different number and type of crystals may be employed in other embodiments of the present disclosure, as well as a magneto-resistive structure well known in the phaco arts.

In use during ophthalmic surgery, an operator grips the surgical handpiece 202 and inserts the needle 210 into a patients' eye (not shown) for emulsifying tissue. The operator generally uses a foot pedal for indicating an operator demand for ultrasonic vibration of the needle 210. When the operator demand is received by the control circuit 204, the control circuit 204 is configured to provide an excitation signal, via cable 206, to the crystal structure 208 included in the surgical handpiece 202. The excitation signal causes ultrasonic vibration of the crystal structure 208. Vibration of the crystal structure 208, in turn, causes ultrasonic vibration of the horn 212 and the needle 210. When the operator no longer desires ultrasonic vibration at the patient's eye, the operator steps off the foot pedal, indicating to the control circuit 204 to cease ultrasonic vibration to the patient's eye. In response, the control circuit 204 ceases outputting the excitation signal to the crystal structure 208.

Upon ceasing the excitation signal to the crystal structure 208, the control circuit 204 is configured to provide a brake signal to the surgical handpiece 202. In this manner, the brake signal is applied substantially immediately after the excitation signal ceases. The brake signal eliminates ultrasonic vibration of the needle 210 within the patient's eye, after the cessation of the excitation signal, which generally reduces a total amount of ultrasonic vibration transmitted to the patient's eye during an ophthalmic surgery.

According to at least the embodiment of FIG. 2, an excitation signal 302 and a brake signal 304 are sinusoidal signals, as shown in FIG. 3. Each of the excitation and the brake signal may, for example, have an amplitude of 200 volts at 28.5 KHz. The brake signal 304, however, is substantially out-of-phase from the excitation signal 302 and preferably 180 degrees out-of-phase, as illustrated by the dotted line. The dotted portion of excitation signal 302 is provided only to illustrate the phase-shift of the brake signal 304. When cessation of the excitation occurs at 306, the excitation signal 302 ceases and the brake signal 304 is generated. Therefore, when as the crystal structure 208 begins to “ring-down” to rest, the brake signal 304 is applied to the crystal structure 208. By being substantially out-of-phase with the excitation signal 302, the brake signal 304 applies a generally opposing force to the vibration of the crystal structure 208. In this manner, any “ring-down” of the crystal structure 208 is reduced or eliminated compared to a surgical handpiece of the prior art.

Further, since the brake signal 304 is out-of-phase from the excitation signal 306, one or more types of energy generated in the surgical handpiece 202 may be prevented by the control circuit 204, potentially resulting in reduced heat generation in the surgical handpiece 202.

In this particular embodiment, the brake signal 304 is about 180 degrees out-of-phase from the excitation signal 302. In other embodiments, other phase-shifts may be employed to eliminate or reduce the vibration of a crystal structure, horn and/or needle after excitation of the crystal structure has ceased. For example, a phase-shift between about 90 degrees and about 270 degrees may be effective to reduce and/or eliminate vibration transmitted to the patient's eye after excitation is ceased. It should be appreciated that various magnitudes and/or frequencies for one or both of an excitation signal and a brake signal may be employed in other embodiments of the present disclosure, depending on safety of the patient, effectiveness of a surgical handpiece, operation of a surgical handpiece, components of a surgical handpiece, type of ophthalmic surgery, etc. Further, a magnitude and/or a frequency of an excitation signal may be different than that of a brake signal. For example, a magnitude of a brake signal may be greater than a magnitude of an excitation signal to increase an opposing force to a crystal structure upon cessation of the excitation signal.

While the excitation signal 302 and brake signal 304 are illustrated as having a sinusoidal waveform in FIG. 3, it should be appreciated that an excitation signal and/or a brake signal can include different waveforms in other embodiments of the present disclosure. Different waveforms may include, e.g., square waveform, triangle waveform, ramp waveform, saw waveform, etc.

The brake signal 304 may be provided to the crystal structure 208 for a fixed time interval sufficient to eliminate vibration of the crystal structure 208 and needle 210. In the particular embodiment of FIGS. 2 and 3, the fixed time interval is preferably matched to the low energy periods of a pulsed waveform known in the art. Accordingly, the brake signal 304 eliminates vibration of the needle 210 before a subsequent excitation of the needle 210, which is dictated by a pulse rate of an excitation signal for a particular ophthalmic surgery. In other embodiments, the brakes signal may be provided for a different fixed time interval or a variable time interval, depending on characteristics and/or operation of a crystal structure, a needle, a horn or another component included in a surgical handpiece. For example, a time interval and/or voltage of a brake signal may be variable and dependent on a current draw by a surgical handpiece when the brake signal is applied.

According to at least one embodiment, a brake signal may be applied after elimination of vibration of a crystal structure and/or a needle such that the crystal structure vibrates according to one or more unique types of patterns. For example, applying a brake signal beyond rest of a crystal structure and/or a needle may result in approximately triangular or sinusoidal amplitude modulated vibration of the needle. Various types, amplitudes, and forms of unique pulsing of a crystal structure may be implemented, depending on requirements of one or more components included in an ophthalmic surgery system and/or an operator.

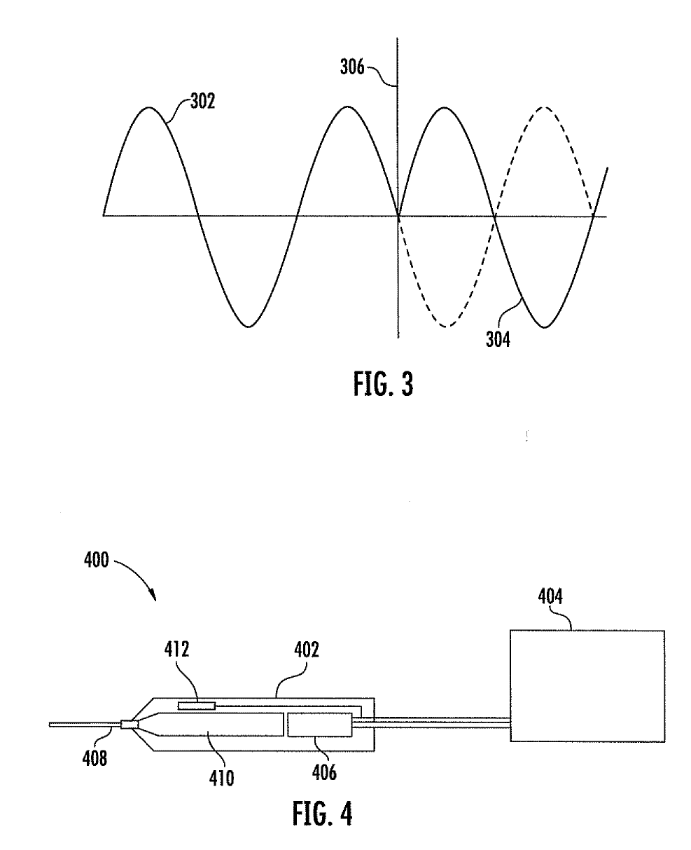

According to one exemplary embodiment of the present disclosure, an ophthalmic surgery system 400 is illustrated in FIG. 4. The ophthalmic surgery system 400 includes a surgical handpiece 402 and a control circuit 404, which is electrically coupled to the surgical handpiece 402. The surgical handpiece 402 includes a piezoelectric crystal structure 406 in mechanical communication with a needle 408, through a horn 410. The control circuit 404 is configured to provide an excitation signal to the crystal structure 406 in response to an operator demand. The surgical handpiece 402 also includes a brake mechanism 412 disposed adjacent to the horn 410. The control circuit is further configured to provide a brake signal to the brake mechanism 412, upon ceasing of the excitation signal to the crystal structure 406. In response to the brake signal, the brake mechanism 412 contacts the horn 410 to eliminate vibration of the horn 410, which in turn eliminates vibration of the needle 408. It should be appreciated that while the brake mechanism 412 is disposed adjacent to the horn 410 for contacting the horn 410, a brake mechanism may be disposed in a different position to contact a different component of a surgical handpiece in other embodiments of the present disclosure. For example, a brake mechanism may be disposed adjacent to a needle of the surgical handpiece in order to contact the needle thereby eliminating vibration of the needle.

The brake mechanism 412 included in the surgical handpiece 402 includes a piezoelectric crystal. It should, however, be appreciated that a brake mechanism may include a different type of crystal and/or mechanical actuator suitable for contacting at least one of a horn, a needle, or a different components of a surgical handpiece to eliminate vibration of the needle included in the surgical handpiece.

Although several aspects of the present disclosure have been described above with reference to phacoemulsification instruments, it should be understood that various aspects of the present disclosure are not limited to phacoemulsification instruments, and can be applied to a variety of other ophthalmic surgical procedures.

By implementing any or all of the teachings described above, a number of benefits and advantages can be attained including improved reliability, reduced down time, elimination or reduction of redundant components or systems, avoiding unnecessary or premature replacement of components or systems, and a reduction in overall system and operating costs.

We claim:

1. A method of eliminating post-excitation vibration provided from a surgical handpiece to a patient's eye during ophthalmic surgery, the surgical handpiece including an ultrasonic vibration structure for generating vibration and a needle for transmitting the vibration into the patient's eye, the method comprising:
exciting the ultrasonic vibration structure and thereby causing the needle to vibrate for emulsifying tissue in the patient's eye;
ceasing the excitation of the ultrasonic vibration structure; and
generating a brake signal to restrain the vibration of the needle, upon ceasing the excitation of the ultrasonic vibration structure, thereby eliminating the vibration provided to the patient's eye after excitation of the ultrasonic vibration structure has ceased.

2. The method of claim 1, wherein the ultrasonic vibration structure is a crystal structure and exciting the crystal structure includes providing an excitation signal to the crystal structure, the brake signal being substantially out-of-phase from the excitation signal.

3. The method of claim 2, wherein the brake signal is about 180 degrees out-of-phase from the excitation signal.

4. The method of claim 2, wherein amplitude of the brake signal is substantially equal to amplitude of the excitation signal.

5. The method of claim 1, wherein outputting the brake signal includes outputting the brake signal to a brake mechanism included in the surgical handpiece assembly, the brake mechanism responsive to the brake signal for contacting one of a horn included in the surgical handpiece and the needle thereby eliminating the vibration of the needle.

6. The method of claim 5 wherein the brake mechanism includes a piezoelectric crystal.

7. An ophthalmic surgery system for eliminating post-excitation vibration provided from a surgical handpiece to a patient's eye during ophthalmic surgery, the ophthalmic surgery system comprising:
a surgical handpiece including an ultrasonic vibration structure and a needle in mechanical communication with the ultrasonic vibration structure whereby excitation of the ultrasonic vibration structure causes the needle to vibrate; and
a control circuit configured to provide an excitation signal to the ultrasonic vibration structure in response to an operator demand, the control circuit being configured to provide a brake signal to the surgical handpiece, upon ceasing the excitation signal to the crystal structure, thereby reducing vibration to a patient's eye after excitation of the crystal structure has ceased.

8. The ophthalmic surgery system of claim 7, wherein the excitation signal and brake signal are sinusoidal signals provided to the ultrasonic vibration structure, the brake signal being substantially out-of-phase from the excitation signal.

9. The ophthalmic surgery system of claim 8, wherein the brake signal is about 180 degrees out-of-phase from the excitation signal.

10. The ophthalmic surgery system of claim 8, wherein amplitude of the brake signal is greater than amplitude of the excitation signal.

11. The ophthalmic surgery system of claim 7, wherein the control circuit is configured to provide the brake signal for a fixed interval.

12. The ophthalmic surgery system of claim 7, wherein the surgical handpiece includes a brake mechanism and a horn, the horn being coupled between the needle and the crystal, the brake mechanism contacting at least one of the horn and the needle, in response to the brake signal, to eliminate vibration of the needle.

13. The method of claim 6 wherein the brake mechanism includes a piezoelectric crystal.
